(19) United States
(12) Patent Application Publication
(10) Pub. No.: US 2016/0030529 A1
(43) Pub. Date: Feb. 4, 2016

(54) TARGETED ELIMINATION OF FACTOR VIII IMMUNE CELLS

(71) Applicant: EMORY UNIVERSITY, Atlanta, GA (US)

(72) Inventors: John S LOLLAR, Decatur, GA (US); John F HEALEY, Snellville, GA (US)

(21) Appl. No.: 14/775,049

(22) PCT Filed: Mar. 13, 2014
(86) PCT No.: PCT/US2014/026203

§ 371 Date: Sep. 11, 2015

Related U.S. Application Data
(60) Provisional application No. 61/778,815, filed on Mar. 13, 2013.

(51) Int. Cl. A61K 38/47; A61K 38/37; C12N 9/24; C07K 14/755

(52) U.S. Cl. CPC A61K 38/47; C07K 14/755; A61K 38/37; C12N 9/2497; C07K 2319/55; C12Y 302/02022

(57) ABSTRACT

This disclosure relates to composition and methods for improving blood clotting in a subject that developed anti-factor VIII antibodies. In certain embodiments, this disclosure contemplates a conjugate comprising a toxin, e.g., ricin, abrin, saporin, coupled to factor VIII or functional variant thereof either through a linking group or as a fusion protein.

### CROSS-REFERENCE TO RELATED APPLICATION

This application claims priority to U.S. Provisional Application No. 61/778,815, filed Mar. 13, 2013, the entire contents of which is hereby incorporated by reference.

### FIELD

This disclosure relates generally to the field of treatments for disorders of blood clotting, and in particular to blood clotting disorders associated with adaptive immune responses to blood clotting factors.

### BACKGROUND

Factor VIII (fVIII) is a cofactor in blood coagulation. After being proteolytically activated, fVIII takes part in a biological cascade that accelerates the conversion of prothrombin to thrombin. Thrombin converts soluble fibrinogen into insoluble fibrin resulting in the coagulation of blood. Typically, fVIII forms a complex with von Willebrand factor VWF (VWF) protecting it from proteolytic degradation.

Hemophilia A is a genetic disease that results from a defective f8 gene. As a result, patients with hemophilia A have low or undetectable levels of fVIII. Blood coagulation in patients with hemophilia is defective, leading to a lifelong bleeding disorder. The coagulation defect in plasma from patients with hemophilia can be corrected by addition of a source of factor VIII. Hemophilia A is typically treated by infusing patients with human recombinant or plasma-derived fVIII. Some patients develop anti-fVIII antibodies. Anti-fVIII antibodies block the procoagulant effect of fVIII. Thus, anti-fVIII antibodies are sometimes referred to as “inhibitors” of blood coagulation. Anti-fVIII antibodies may occur in individuals with hemophilia A or in non-hemophilic individuals who develop auto-immunity to their own fVIII. Both allo- and auto-immune produced anti-fVIII antibodies can be life threatening. There is a need for improved therapies to reduce the incidence of anti-fVIII antibodies.

Volkman et al. report selectively eliminating human antigen-specific B cell responses by treating cells in vitro with antigen covalently linked to a cell toxin. See J Exp Med, 1982, 156(2):634-9. Arndt & Thesen report targeting antigen receptors with conjugates of antigens and toxin to eliminate antigen-reactive cells. Scand. J. Immunol, 1985, 22:489-494. See also Brozek et al., J Immuno, 1984, 132(3):1144-1150; Frier et al., Leukemia & Lymphoma, 2003, 44(4):681-689, and Messerschmidt & Heilmann, J Immunol Methods, 2013, 387(1-2): 167-72.

Meeks et al. report that a determinant of the immunogenicity of fVIII is independent of its procoagulant function. See Blood, 2012, 120(12) 2512. See also Markovitz et al., Blood, 2013, doi:10.1182, entitled “The diversity of the immune response to the A2 domain of human factor VIII.”

References cited herein are not an admission of prior art.

### SUMMARY

This disclosure relates to composition and methods for improving blood clotting in a subject that has developed anti-factor VIII antibodies or preventing anti-fVIII antibodies in a hemophilia A patient who is exposed to fVIII. When a patient with naïve or memory B-cells with surface immunoglobulin that binds fVIII (fVIII-specific sIg) is treated with recombinant factor VIII, the B cells are stimulated, leading the production of anti-fVIII antibodies. These antibodies inhibit fVIII activity and, as a result, blood clotting is prevented. In certain embodiments, this disclosure contemplates a conjugate comprising a toxin coupled to factor VIII or functional variant thereof either through a linking group or as a fusion protein. Examples of suitable toxins are ricin, abrin, saporin, or derivatives thereof.

Although it is not intended that certain embodiments of the disclosure be limited by any particular mechanism, it is believed that naïve and memory B cells bind to the toxin-factor VIII conjugate because memory and naïve B cells contain fVIII-specific sIgs. The toxin is internalized and disables or kills the B cells, decreasing the formation of anti-factor VIII antibodies and allowing administration of factor VIII to mediated blood clotting in a subject.

In certain embodiments, the disclosure relates to compositions comprising a conjugate containing a factor VIII polypeptide or variant thereof and a toxin. In certain embodiments, the toxin is a polypeptide, and in specific embodiments is saporin, ricin, or abrin, or a derivative thereof. In certain embodiments, the toxin is a ribosome inactivating protein. In certain embodiments, the factor VIII polypeptide is linked to the toxin by a linking group such as a linking group comprising a biotin binding protein such as avidin or streptavidin. In other embodiments, the conjugate is a direct conjugate between the fVIII or variant thereof and the toxin, such as through a disulfide bond linkage or an amine-thiol bond. In specific embodiments, the conjugate is an amine-thiol conjugate.

In certain embodiments, the conjugate is a recombinant polypeptide. In certain embodiments, the disclosure relates to nucleic acids encoding a recombinant polypeptide disclosed herein. In certain embodiments, the disclosure relates to expression vectors comprising a nucleic acid disclosed herein. In certain embodiments, the disclosure relates to expression systems comprising a vector disclosed herein.

In certain embodiments, the disclosure relates to methods of improving blood clotting comprising administering a conjugate disclosed herein to a subject that developed or is at risk of developing anti-factor VIII antibodies wherein administration is in an effective amount to prevent an adaptive immune response to factor VIII. In certain embodiments, factor VIII is administered in combination with or after administering the conjugate. In certain embodiments, the subject is diagnosed with Hemophilia A.

### BRIEF DESCRIPTION OF THE FIGURES

FIG. 1 shows data on the characterization of inactive fVIII constructs. (A) SDS-PAGE of wt fVIII, R372A/R1689A fVIII, and V634M fVIII with and without exposure to thrombin (factor IIa). MW STDS indicates molecular weight standards; SC, single chain fVIII; HC, fVIII heavy chain (A1-A2 domains); LC, fVIII light chain; LCIIa, thrombin-cleaved light chain; A1, A1 domain; and A2, A2 domain. (B) Thrombin generation in fVIII-deficient plasma reconstituted with 2 μg/mL wt fVIII, R372A/R1689A fVIII, or V634M fVIII. (C) Binding of wt fVIII, R372A/R1689A fVIII, or V634M fVIII to immobilized VWF in the presence or absence of exposure to thrombin detected by ELISA as described in “Binding of fVIII to VWF.” (D) ELISA of binding of wt fVIII, R372A/R1689A fVIII, or V634M fVIII to immobilized domain-specific anti-fVIII capture mAbs 2-116 (A1), 1D4 (A2), 2-54 (A2), 2-93 (A2), 4A4 (A2), 2-113 (A3), G38 (A3), 5G12, 2A9 (C1), 1-109 (C2), and I14 (C2, negative control). Biotinylated I14 was used as the detection antibody. (E) Clearance of 1 μg of wt fVIII, R372A/R1689A fVIII, or V634M fVIII after tail-vein injection in FVIII−/− mice. Errors represent sample SDs.

FIG. 2 shows data on the antibody response to low-dose wt fVIII and R372A/R1689A fVIII in fVIII−/− mice. FVIII−/− mice were injected with 6 weekly doses of wt fVIII (n=25) or R372A/R1689A fVIII (n=25) of 0.2 μg, followed by 2 additional doses of 0.5 μg. One week after the last dose, plasma was collected for measurement of (A) total anti-fVIII IgG by ELISA and (B) fVIII inhibitor titers by the Bethesda assay. The difference in ELISA titers and inhibitor titers between the 2 groups was significant (P=0.03 and 0.02, Mann-Whitney test). (C) The binding of biotinylated ESH4, a mAb that recognizes the classic C2 domain epitope overlapping a VWF binding site, was measured in the absence (filled circles) and presence (open triangles) of a 1/24 dilution of a high-titer plasma from a mouse immunized with R372A/R1689A fVIII. *P<0.05.

FIG. 3 shows data on the dose-dependent immunogenicity of wt fVIII, R372A/R1689A fVIII, and V634M fVIII in fVIII−/− mice. Cohorts (n=9-10) of fVIII−/− were injected with 4 weekly doses of fVIII construct (0.5, 1.0, 1.5, or 2.0 μg) weekly, followed by a single boost dose at twice the weekly dose. One week after the last dose plasma was collected for both (A) anti-fVIII ELISA titers and (B) fVIII inhibitor titers. Graphs show medians and interquartile ranges.

FIG. 4 shows data on the antibody response to wt fVIII, R372A/R1689A fVIII, or V634M fVIII in fVIII−/−/VWF−/− mice. FVIII−/−/VWF−/− mice were injected with 6 weekly doses of wt fVIII (n=13), R372A/R1689A fVIII (n=14), or V634M fVIII (n=13) at 0.6 μg, followed by 2 weekly doses of 1.5 μg. One week after the last dose plasma was collected for both (A) anti-fVIII ELISA titers and (B) fVIII inhibitor titers. The differences in anti-fVIII antibody and fVIII inhibitor titers between wt fVIII and R372A/R1689A fVIII and V634M fVIII were not statistically significant.

FIG. 5 shows a table with data from bioassays of purified fVIII constructs with comparison to fVIII-deficient plasma. * fVIII activity was determined by 1-stage coagulation assay as described in “FVIII bioassays.”† fVIII-dependent intrinsic factor Xase activity was measured as described in “FVIII bioassays” and expressed relative to wt fVIII. ‡ Thrombin generation assays were performed as described in “FVIII bioassays.” Errors represent sample SDs derived from triplicates from each of 2 independent experiments.

FIG. 6 shows a table with data from comparative immunogenicity of wt fVIII, R372A/R1689A fVIII, and R372A/R1689A fVIII in fVIII−/− mice. * P<0.05 when compared with wt fVIII.

FIG. 7 illustrates an experiment on the in vivo effect of a saporin-fVIII conjugate.

### DETAILED DISCUSSION

Before the present disclosure is described in greater detail, it is to be understood that this disclosure is not limited to particular embodiments described, and as such may, of course, vary. It is also to be understood that the terminology used herein is for the purpose of describing particular embodiments only, and is not intended to be limiting, since the scope of the present disclosure will be limited only by the appended claims.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Prior to describing the various embodiments, the following definitions are provided and should be used unless otherwise indicated.

“Subject” refers any animal, preferably a human patient, livestock, rodent, monkey or domestic pet. A subject that has developed an immune response or that has developed anti-fVIII antibodies can be assessed by measuring antibodies, typically using an ELISA or similar kit, or can be identified by reduced clotting or increased clotting time in response to administration of commercially available fVIII protein in blood or blood samples of the subject.

As used herein, the terms “prevent” and “preventing” include the prevention of the recurrence, spread or onset. It is not intended that the present disclosure be limited to complete prevention. In some embodiments, the onset is delayed, or the severity of the disease or symptom is reduced.

As used herein, the terms “treat” and “treating” are not limited to the case where the subject (e.g., patient) is cured and the disease is eradicated. Rather, embodiments, of the present disclosure also contemplate treatment that merely reduces symptoms, and/or delays disease progression.

As used herein, the term “combination with” when used to describe administration with an additional treatment means that the agent may be administered prior to, together with, or after the additional treatment, or a combination thereof.

The term “a nucleic acid sequence encoding” a specified polypeptide refers to a nucleic acid sequence comprising the coding region of a gene or in other words the nucleic acid sequence which encodes a gene product. The coding region may be present in either cDNA, genomic DNA, or RNA form. When present in a DNA form, the oligonucleotide, polynucleotide, or nucleic acid may be single-stranded (i.e., the sense strand) or double-stranded. Suitable control elements such as enhancers/promoters, splice junctions, polyadenylation signals, etc. may be placed in close proximity to the coding region of the gene if needed to permit proper initiation of transcription and/or correct processing of the primary RNA transcript. Alternatively, the coding region utilized in the expression vectors of the present invention may contain endogenous enhancers/promoters, splice junctions, intervening sequences, polyadenylation signals, etc. or a combination of both endogenous and exogenous control elements.

The terms “vector” or “ expression vector ” refer to a recombinant nucleic acid containing a desired coding sequence and appropriate nucleic acid sequences necessary for the expression of the operably linked coding sequence in a particular host organism or expression system, e.g., cellular or cell-free. Nucleic acid sequences necessary for expression in prokaryotes usually include a promoter, an operator (optional), and a ribosome binding site, often along with other sequences. Eukaryotic cells are known to utilize promoters, enhancers, and termination and polyadenylation signals.

Protein “expression systems” refer to in vivo and in vitro (cell free) systems. Systems for recombinant protein expression typically utilize cells transfecting with a DNA expression vector that contains the template. The cells are cultured under conditions such that they translate the desired protein. Expressed proteins are extracted for subsequent purification. In vivo protein expression systems using prokaryotic and eukaryotic cells are well known. In some cases, a eukaryotic expression system will be necessary to express a protein, and in certain cases, such as when a protein is toxic to eukaryotic cells, a prokaryotic expression system may be desirable. Also, some proteins are recovered using denaturants and protein-refolding procedures. In vitro (cell-free) protein expression systems typically use translation-compatible extracts of whole cells or compositions that contain components sufficient for transcription, translation and optionally post-translational modifications such as RNA polymerase, regulatory protein factors, transcription factors, ribosomes, tRNA cofactors, amino acids and nucleotides. In the presence of an expression vectors, these extracts and components can synthesize proteins of interest. Cell-free systems typically do not contain proteases and enable labeling of the protein with modified amino acids. Some cell free systems incorporated encoded components for translation into the expression vector. See, e.g., Shimizu et al., Cell-free translation reconstituted with purified components, 2001, Nat. Biotechnol., 19, 751-755 and Asahara & Chong, Nucleic Acids Research, 2010, 38(13): e141, both hereby incorporated by reference in their entirety.

The term “recombinant” when made in reference to a nucleic acid molecule refers to a nucleic acid molecule which is comprised of segments of nucleic acid joined together by means of molecular biological techniques. The term “recombinant” when made in reference to a protein or a polypeptide refers to a protein molecule which is expressed using a recombinant nucleic acid molecule.

The terms “in operable combination”, “in operable order” and “operably linked” refer to the linkage of nucleic acid sequences in such a manner that a nucleic acid molecule capable of directing the transcription of a given gene and/or the synthesis of a desired protein molecule is produced. The term also refers to the linkage of amino acid sequences in such a manner so that a functional protein is produced.

The term “regulatory element” refers to a genetic element which controls some aspect of the expression of nucleic acid sequences. For example, a promoter is a regulatory element which facilitates the initiation of transcription of an operably linked coding region. Other regulatory elements are splicing signals, polyadenylation signals, termination signals, etc.

Transcriptional control signals in eukaryotes comprise “promoter” and “enhancer” elements. Promoters and enhancers consist of short arrays of DNA sequences that interact specifically with cellular proteins involved in transcription (Maniatis, et al., Science 236:1237, 1987). Promoter and enhancer elements have been isolated from a variety of eukaryotic sources including genes in yeast, insect, mammalian and plant cells. Promoter and enhancer elements have also been isolated from viruses and analogous control elements, such as promoters, are also found in prokaryotes. The selection of a particular promoter and enhancer depends on the cell type used to express the protein of interest. Some eukaryotic promoters and enhancers have a broad host range while others are functional in a limited subset of cell types (for review, see Voss, et al., Trends Biochem. Sci., 11:287, 1986; and Maniatis, et al., supra 1987).

The terms “promoter element,” “promoter,” or “promoter sequence” as used herein, refer to a DNA sequence that is located at the 5′ end (i.e., precedes) the protein coding region of a DNA polymer. The location of most promoters known in nature precedes the transcribed region. The promoter functions as a switch, activating the expression of a gene. If the gene is activated, it is said to be transcribed, or participating in transcription. Transcription involves the synthesis of mRNA from the gene. The promoter, therefore, serves as a transcriptional regulatory element and also provides a site for initiation of transcription of the gene into mRNA. The term “cell type specific” as applied to a promoter refers to a promoter which is capable of directing selective expression of a nucleotide sequence of interest in a specific type of cell in the relative absence of expression of the same nucleotide sequence of interest in a different type of cell within the same tissue. Promoters may be constitutive or regulatable. The term “constitutive” when made in reference to a promoter means that the promoter is capable of directing transcription of an operably linked nucleic acid sequence in the absence of a stimulus (e.g., heat shock, chemicals, light, etc.). Typically, constitutive promoters are capable of directing expression of a transgene in substantially any cell and any tissue. In contrast, a “regulatable” or “inducible” promoter is one which is capable of directing a level of transcription of an operably linked nuclei acid sequence in the presence of a stimulus (e.g., heat shock, chemicals, light, etc.) which is different from the level of transcription of the operably linked nucleic acid sequence in the absence of the stimulus.

The enhancer and/or promoter may be “endogenous” or “exogenous” or “heterologous.” An “endogenous” enhancer or promoter is one that is naturally linked with a given gene in the genome. An “exogenous” or “heterologous” enhancer or promoter is one that is placed in juxtaposition to a gene by means of genetic manipulation (i.e., molecular biological techniques) such that transcription of the gene is directed by the linked enhancer or promoter. For example, an endogenous promoter in operable combination with a first gene can be isolated, removed, and placed in operable combination with a second gene, thereby making it a “heterologous promoter” in operable combination with the second gene.

Efficient expression of recombinant nucleic acid sequences in eukaryotic cells requires expression of signals directing the efficient termination and polyadenylation of the resulting transcript. Transcription termination signals are generally found downstream of the polyadenylation signal and are typically a few hundred nucleotides in length. The term “poly(A) site” or “poly(A) sequence” as used herein denotes a DNA sequence which directs both the termination and polyadenylation of the nascent RNA transcript. Efficient polyadenylation of the recombinant transcript is desirable, as transcripts lacking a poly(A) tail are unstable and are rapidly degraded. The poly(A) signal utilized in an expression vector may be “heterologous” or “endogenous.” An endogenous poly(A) signal is one that is found naturally at the 3′ end of the coding region of a given gene in the genome. A heterologous poly(A) signal is one which has been isolated from one gene and positioned 3′ to another gene.

Sequence “identity” refers to the number of exactly matching residues (expressed as a percentage) in a sequence alignment between two sequences of the alignment. As used herein, percentage identity of an alignment is calculated using the number of identical positions divided by the greater of the shortest sequence or the number of equivalent positions excluding overhangs wherein internal gaps are counted as an equivalent position. For example the polypeptides GGGGGG and GGGGT have a sequence identity of 4 out of 5 or 80%. For example, the polypeptides GGGPPP and GGGAPPP have a sequence identity of 6 out of 7 or 85%.

Percent “similarity” is used to quantify the similarity between two sequences of the alignment. This method is identical to determining the identity except that certain amino acids do not have to be identical to have a match. Amino acids are classified as matches if they are among a group with similar properties according to the following amino acid groups: Aromatic—F Y W; hydrophobic—A V I L; Charged positive: R K H; Charged negative—D E; Polar—S T N Q.

The terms “variant” or “derivative” when used in reference to a polypeptide refer to an amino acid sequence that differs by one or more amino acids from another, usually related polypeptide. The variant may have “conservative” changes, wherein a substituted amino acid has similar structural or chemical properties. One type of conservative amino acid substitutions refers to the interchangeability of residues having similar side chains. For example, a group of amino acids having aliphatic side chains is glycine, alanine, valine, leucine, and isoleucine; a group of amino acids having aliphatic-hydroxyl side chains is serine and threonine; a group of amino acids having amide-containing side chains is asparagine and glutamine; a group of amino acids having aromatic side chains is phenylalanine, tyrosine, and tryptophan; a group of amino acids having basic side chains is lysine, arginine, and histidine; and a group of amino acids having sulfur-containing side chains is cysteine and methionine. Preferred conservative amino acids substitution groups are: valine-leucine-isoleucine, phenylalanine-tyrosine, lysine-arginine, alanine-valine, and asparagine-glutamine. More rarely, a variant may have “non-conservative” changes (e.g., replacement of a glycine with a tryptophan). Similar minor variations may also include amino acid deletions or insertions (in other words, additions), or both. Guidance in determining which and how many amino acid residues may be substituted, inserted or deleted without abolishing biological activity may be found using computer programs well known in the art, for example, DNAStar software. Variants can be tested in functional assays. Certain variants have less than 10%, and preferably less than 5%, and still more preferably less than 2% changes (whether substitutions, deletions, and so on). In certain embodiments, the variant or derivative retains activity of the original molecule.

### Conjugates of Factor VIII and Toxins

In certain embodiment, conjugates disclosed herein comprise a toxin and fVIII or a fragment or derivative thereof that binds fVIII-specific sIgs on the surface of B cells. The conjugates can be produced by chemical conjugation of two peptides, one including a toxin peptide sequence and the other a fVIII or derivative sequence. In other embodiments, the conjugate is generated by expression of fusion proteins in which, for example, a DNA encoding a toxin with or without a linker region to DNA encoding fVIII. The conjugates may also be produced by chemical coupling, e.g., through disulfide bonds between cysteine residues present in or added to the components, or through amide bonds, or other suitable bonds. Ionic bonding and other hydrogen bonding linkages are also contemplated, such as ligand receptor interactions, e.g., biotin/avidin or streptavidin.

In certain embodiments, the linker is a peptide or a non-peptide. When fusion proteins are contemplated, the linker is selected such that the resulting nucleic encodes a fusion protein that binds to and is internalized by B cells that express fVIII-specific sIgs. It is also contemplated that several linkers can be joined in order to employ the advantageous properties of each linker. In such instance, the linker portion of conjugate may contain more than 1 to 50 amino acid residues. The number of residues is not important as long as the resulting fusion protein binds to an anti-fVIII antibody and the B cell internalizes the linked toxin.

A typical conjugate is a fusion protein containing fVIII or fragment connected to a cellular toxin via a peptide linker. Conjugates that contain fVIII linked, either directly or via a linker, to one or more toxins are provided. In particular, conjugates provided herein contain the following components: (fVIII)n, (L)q, and (toxin)m in which at least one fVIII, such as a full length fVIII, or an effective fragment thereof, is linked directly or via one or more linkers (L) to at least one toxin. L refers to a linker. Any suitable association among the elements of the conjugate is contemplated as long as the resulting conjugates interact with an anti-fVIII antibody on a B cell such that internalization of an associated toxin is affected.

The variables n and m are integers of 1 or greater and q is 0 or any integer. The variables n, q and m are selected such that the resulting conjugate interacts with the anti-fVIII antibody and a toxin is internalized by a B cell to which it has been targeted.

It is understood that the above description does not represent the order in which each component is linked or the manner in which each component is linked. The fVIII and toxin may be linked in any order and through any appropriate linkage, as long as the resulting conjugate binds to an anti-fVIII antibody and internalizes the toxin in B cells bearing the antibody

FVIII is typically a single-chain precursor of approximately 270-330 kD with a 19 residue propeptide signal comprised of a 19 amino acids and a domain structure A1-A2-B-ap-A3-C1-C2, where ap refers to an activation peptide that is released during proteolytic activation. When purified from plasma (e.g., “plasma-derived” or “plasmatic”), fVIII is composed of a heavy chain (A1-A2-B) and a light chain (ap-A3-C1-C2). The molecular mass of the light chain is about 80 kD whereas, due to proteolysis within the B domain, the heavy chain is in the range of about 90-220 kD.

A typical fVIII amino acid sequence including the propeptide is provided as SEQ ID NO: 1. MQIELSTCFF LCLLRFCFSA TRRYYLGAVE LSWDYMQSDL GELPVDARFP PRVPKSFPFN TSVVYKKTLF VEFTDHLFNI AKPRPPWMGL LGPTIQAEVY DTVVITLKNM ASHPVSLHAV GVSYWKASEG AEYDDQTSQR EKEDDKVFPG GSHTYVWQVL KENGPMASDP LCLTYSYLSH VDLVKDLNSG LIGALLVCRE GSLAKEKTQT LHKFILLFAV FDEGKSWHSE TKNSLMQDRD AASARAWPKM HTVNGYVNRS LPGLIGCHRK SVYWHVIGMG TTPEVHSIFL EGHTFLVRNH RQASLEISPI TFLTAQTLLM DLGQFLLFCH ISSHQHDGME AYVKVDSCPE EPQLRMKNNE EAEDYDDDLT DSEMDVVRFD DDNSPSFIQI RSVAKKHPKT WVHYIAAEEE DWDYAPLVLA PDDRSYKSQY LNNGPQRIGR KYKKVRFMAY TDETFKTREA IQHESGILGP LLYGEVGDTL LIIFKNQASR PYNIYPHGIT DVRPLYSRRL PKGVKHLKDF PILPGEIFKY KWTVTVEDGP TKSDPRCLTR YYSSFVNMER DLASGLIGPL LICYKESVDQ RGNQIMSDKR NVILFSVFDE NRSWYLTENI QRFLPNPAGV QLEDPEFQAS NIMHSINGYV FDSLQLSVCL HEVAYWYILS IGAQTDFLSV FFSGYTFKHK MVYEDTLTLF PFSGETVFMS MENPGLWILG CHNSDFRNRG MTALLKVSSC DKNTGDYYED SYEDISAYLL SKNNAIEPRS FSQNSRHPST RQKQFNATTI PENDIEKTDP WFAHRTPMPK IQNVSSSDLL MLLRQSPTPH GLSLSDLQEA KYETFSDDPS PGAIDSNNSL SEMTHFRPQL HHSGDMVFTP ESGLQLRLNE KLGTTAATEL KKLDFKVSST SNNLISTIPS DNLAAGTDNT SSLGPPSMPV HYDSQLDTTL FGKKSSPLTE SGGPLSLSEE NNDSKLLESG LMNSQESSWG KNVSSTESGR LFKGKRAHGP ALLTKDNALF KVSISLLKTN KTSNNSATNR KTHIDGPSLL IENSPSVWQN ILESDTEFKK VTPLIHDRML MDKNATALRL NHMSNKTTSS KNMEMVQQKK EGPIPPDAQN PDMSFFKMLF LPESARWIQR THGKNSLNSG QGPSPKQLVS LGPEKSVEGQ NFLSEKNKVV VGKGEFTKDV GLKEMVFPSS RNLFLTNLDN LHENNTHNQE KKIQEEIEKK ETLIQENVVL PQIHTVTGTK NFMKNLFLLS TRQNVEGSYD GAYAPVLQDF RSLNDSTNRT KKHTAHFSKK GEEENLEGLG NQTKQIVEKY ACTTRISPNT SQQNFVTQRS KRALKQFRLP LEETELEKRI IVDDTSTQWS KNMKHLTPST LTQIDYNEKE KGAITQSPLS DCLTRSHSIP QANRSPLPIA KVSSFPSIRP IYLTRVLFQD NSSHLPAASY RKKDSGVQES SHFLQGAKKN NLSLAILTLE MTGDQREVGS LGTSATNSVT YKKVENTVLP KPDLPKTSGK VELLPKVHIY QKDLFPTETS NGSPGHLDLV EGSLLQGTEG AIKWNEANRP GKVPFLRVAT ESSAKTPSKL LDPLAWDNHY GTQIPKEEWK SQEKSPEKTA FKKKDTILSL NACESNHAIA AINEGQNKPE IEVTWAKQGR TERLCSQNPP VLKRHQREIT RTTLQSDQEE IDYDDTISVE MKKEDFDIYD EDENQSPRSF QKKTRHYFIA AVERLWDYGM SSSPHVLRNR AQSGSVPQFK KVVFQEFTDG SFTQPLYRGE LNEHLGLLGP YIRAEVEDNI MVTFRNQASR PYSFYSSLIS YEEDQRQGAE PRKNFVKPNE TKTYFWKVQH HMAPTKDEFD CKAWAYFSDV DLEKDVHSGL IGPLLVCHTN TLNPAHGRQV TVQEFALFFT IFDETKSWYF TENMERNCRA PCNIQMEDPT FKENYRFHAI NGYIMDTLPG LVMAQDQRIR WYLLSMGSNE NIHSIHFSGH VFTVRKKEEY KMALYNLYPG VFETVEMLPS KAGIWRVECL IGEHLHAGMS TLFLVYSNKC QTPLGMASGH IRDFQITASG QYGQWAPKLA RLHYSGSINA WSTKEPFSWI KVDLLAPMII HGIKTQGARQ KFSSLYISQF IIMYSLDGKK WQTYRGNSTG TLMVFFGNVD SSGIKHNIFN PPIIARYIRL HPTHYSIRST LRMELMGCDL NSCSMPLGME SKAISDAQIT ASSYFTNMFA TWSPSKARLH LQGRSNAWRP QVNNPKEWLQ VDFQKTMKVT GVTTQGVKSL LTSMYVKEFL ISSSQDGHQW TLFFQNGKVK VFQGNQDSFT PVVNSLDPPL LTRYLRIHPQ SWVHQIALRM EVLGCEAQDL Y.

When the expressed polypeptide is translocated into the lumen of the endoplasmic reticulum, however, the signal sequence is cleaved, resulting in a second sequence. This second sequence, herein provided as SEQ ID NO:2, lacks the leading 19 amino acids is conventionally used by researchers to assign a numeric location (e.g., Arg372) to a given amino acid residue of fVIII. Thus, unless specifically noted, all assignments of a numeric location of an amino acid residue as provided herein are based on SEQ ID NO:2.

Various in vitro assays have been devised to determine the potential efficacy of recombinant fVIII (rfVIII) as a therapeutic medicine. These assays mimic the in vivo effects of endogenous fVIII. In vitro thrombin treatment of fVIII results in a rapid increase and subsequent decrease in its procoagulant activity, as measured by in vitro assay. This activation and inactivation coincides with specific limited proteolysis both in the heavy and the light chains, which alter the availability of different binding epitopes in fVIII, e.g. allowing fVIII to dissociate from VWF and bind to a phospholipid surface or altering the binding ability to certain monoclonal antibodies.

Herein, the term “factor VIII” or “fVIII” refers to any fVIII molecule which exhibits biological activity that is associated with wt fVIII. In one embodiment, the fVIII molecule is full-length factor VIII. fVIII may contain amino acid deletions at various sites between or within the domains A1-A2-B-A3-C1-C2. The molecule may also be an analog of wt fVIII wherein one or more amino acid residues have been replaced by site-directed mutagenesis.

According to the present disclosure, the term “recombinant factor VIII” (rfVIII) may include any rfVIII, heterologous or naturally occurring, obtained via recombinant DNA technology, or a biologically active derivative thereof. In certain embodiments, the term encompasses proteins as described above obtained from nucleic acids encoding a rfVIII. Such nucleic acids include, for example and without limitation, genes, pre-mRNAs, mRNAs, polymorphic variants, alleles, synthetic and naturally-occurring mutants. Proteins embraced by the term rfVIII include, for example and without limitation, those proteins and polypeptides described herein, proteins encoded by a nucleic acid described above, interspecies homologs and other polypeptides that have an amino acid sequence that has greater than about 60%, about 65%, about 70%, about 75%, about 80%, about 85%, about 90%, about 91%, about 92%, about 93%, about 94%, about 95%, about 96%, about 97%, about 98% or about 99% or greater amino acid sequence identity or similarity, over a region of at least about 25, about 50, about 100, about 200, about 300, about 400, or more amino acids.

Variant (or analog) polypeptides include insertion variants, wherein one or more amino acid residues are added to an fVIII amino acid sequence. Insertions may be located at either or both termini of the protein, and/or may be positioned within internal regions of the fVIII amino acid sequence. Insertion variants, with additional residues at either or both termini, include for example, fusion proteins and proteins including amino acid tags or other amino acid labels. In one aspect, the fVIII molecule may optionally contain an N-terminal Met, especially when the molecule is expressed recombinantly in a bacterial cell such as E. coli. In deletion variants, one or more amino acid residues in a fVIII polypeptide as described herein are removed. Deletions can be effected at one or both termini of the fVIII polypeptide, and/or with removal of one or more residues within the fVIII amino acid sequence. Deletion variants, therefore, include all fragments of a fVIII polypeptide sequence.

Within any of the embodiments disclosed herein, fVIII may be derived from human plasma, or produced by recombinant engineering techniques, as described in U.S. Pat. No. 4,757,006; U.S. Pat. No. 5,733,873; U.S. Pat. No. 5,198,349; U.S. Pat. No. 5,250,421; U.S. Pat. No. 5,919,766; EP 306 968.

Within any of the embodiments disclosed herein, the toxin is any agent for which targeted delivery by fVIII prevents B cells from promoting an adaptive immune response. Contemplated toxins include, but are not limited to, a cytotoxic agent, a radioactive agent, a, a ribosome inactivating protein (RIP), aquatic-derived cytotoxins, inhibitors of DNA, RNA or protein synthesis, metabolic inhibitors, DNA cleaving molecules or the like. Specific examples include gelonin, saporin, abrin, Pseudomonas exotoxin, light activated porphyrins, Shiga toxin, shiga-A1, ricin A chain, E. coli-produced colicins, shiga-like toxins, maize RIP, gelonin, diphtheria toxin, diphtheria toxin A chain, trichosanthin, tritin, pokeweed antiviral protein (PAP), mirabilis antiviral protein (MAP), Dianthins 32 and 30, monordin, bryodin, cytotoxically active fragments of these cytotoxins and toxins, and other toxins, or a drug, such as methotrexate. In certain embodiments, the toxin comprises a domain of the Ribosome inactivating protein (RIP) superfamily c108249.

In certain specific embodiments, the toxin comprises the saporin chain A (SEQ ID NO: 3)

VTSITLDLVN PTAGQYSSFV DKIRNNVKDP NLKYGGTDIA
VIGPPSKEKF LRINFQSSRG TVSLGLKRDN LYVVAYLAMD
NTNVNRAYYF KSEITSAELT ALFPEATTAN QKALEYTEDY
QSIEKNAQIT QGDKSRKELG LGIDLLLTFM EAVNKKARVV
KNEARFLLIA IQMTAEVARF RYIQNLVTKN FPNKFDSDNK
VIQFEVSWRK ISTAIYGDAK NGVFNKDYDF GFGKVRQVKD
LQMGLLMYLG KPK.

Proteins embraced by the term toxin include, for example and without limitation, those proteins and polypeptides described herein interspecies homologs and other polypeptides that have an amino acid sequence that has greater than about 60%, about 65%, about 70%, about 75%, about 80%, about 85%, about 90%, about 91%, about 92%, about 93%, about 94%, about 95%, about 96%, about 97%, about 98% or about 99% or greater amino acid sequence identity or similarity, over a region of at least about 25, about 50, about 100, about 200, about 300, about 400, or more amino acids.

In certain embodiments, the disclosure relates to fVIII-toxin conjugates comprising polyethylene glycol (PEG). The PEGylation process attaches repeating units of polyethylene glycol (PEG) to a polypeptide drug. PEGylation of molecules can lead to increased resistance of drugs to enzymatic degradation, increased half-life in vivo, reduced dosing frequency, decreased immunogenicity, increased physical and thermal stability, increased solubility, increased liquid stability, and reduced aggregation.

In certain embodiments, the disclosure relates to a proteinaceous construct comprising fVIII-toxin conjugate comprising a water-soluble polymer. In certain embodiments, the water-soluble polymer comprises a polyalkylene oxide, polyvinyl pyrrolidone, polyvinyl alcohol, polyoxazoline, a poly acryloylmorpholine or a carbohydrate, such as polysialic acid (PSA) or dextran. U.S. Pat. No. 8,071,728 reports a Factor VIII molecule and water soluble polymer is attached to the factor VIII via one or more carbohydrate moieties.

In certain embodiments, the disclosure contemplates a composition comprising a purified complex wherein the fVIII-toxin conjugate binds VWF. U.S. Pat. No. 6,307,032 reports a purified complex comprising the components factor VIII and VWF.

### A Determinant of the Immunogenicity of Factor VIII is Independent of its Procoagulant Function

FVIII inhibitor formation in patients with hemophilia A and in mice with hemophilia A (fVIII−/−) is a MHC class II T cell-dependent process. During T cell-dependent antibody formation, T-cell receptors on naïve T cells recognize antigen bound to MHC class II molecules on the surface of antigen presenting cells (APCs), including dendritic cells (DCs), macrophages, and B cells, that are present in secondary lymphoid organs (e.g., lymph nodes and the spleen). Antigen presentation when combined with appropriate costimulatory signals results in T-cell activation and proliferation and differentiation into T-helper cells.

Because fVIII is an immunologically foreign protein or an altered self-protein in patients with severe hemophilia A and fVIII−/− mice, it may not seem surprising that it produces an antibody response. However, it usually is difficult to raise antibodies to a foreign protein without using adjuvants, especially when the protein is delivered by intravenous administration. In a direct comparison of the immunogenicity of equal doses of adjuvant-free ovalbumin and human fVIII in fVIII−/− mice, anti-fVIII antibody titers were ˜100-fold higher than anti-ovalbumin titers. Model monomeric protein immunogens such as ovalbumin or lysozyme typically are given with adjuvants with a dose >50-fold higher than the adjuvant-free doses of ˜10 μg/kg or less that are required to produce fVIII inhibitors in patients with hemophilia A and fVIII−/− mice. In addition, although the concentration of fVIII in plasma is lower than all the other coagulation factors, it is the most commonly targeted coagulation factor in autoimmunity. Thus, fVIII evidently is an unusually immunogenic protein. FVIII circulates noncovalently bound to VWF, which must be considered as a possible factor in the immunogenicity of fVIII.

To address the immunogenicity of fVIII independent of its procoagulant and potentially proinflammatory function and the role of VWF in the process, the immune response of 2 inactive, conformationally intact recombinant B domain-deleted (BDD) fVIII molecules to wild-type (wt) in fVIII−/− and fVIII−/−/VWF−/− mice were compared. R372A/R1689A fVIII lacks cleavage sites that are recognized by thrombin and factor Xa. Cleavage at R372 between the A1 and A2 domains of fVIII is necessary for production of factor IXa cofactor activity that is the basis of the procoagulant function of fVIII. Cleavage at R1689 in the light chain of fVIII leads to the dissociation of activated fVIII from VWF, binding to activated platelets and assembly of the intrinsic pathway factor X-activating complex. Because R372A/R1689A fVIII is not released from VWF, it should not localize to procoagulant sites or promote thrombin formation. V634M fVIII contains a single substitution in the A2 domain that leads to a profound loss of procoagulant activity. It has normal proteolytic recognition sites and dissociates from VWF on exposure to thrombin. Thus, it should not contribute to thrombin production, but, unlike R372A/R1689A fVIII, it should localize to procoagulant sites.

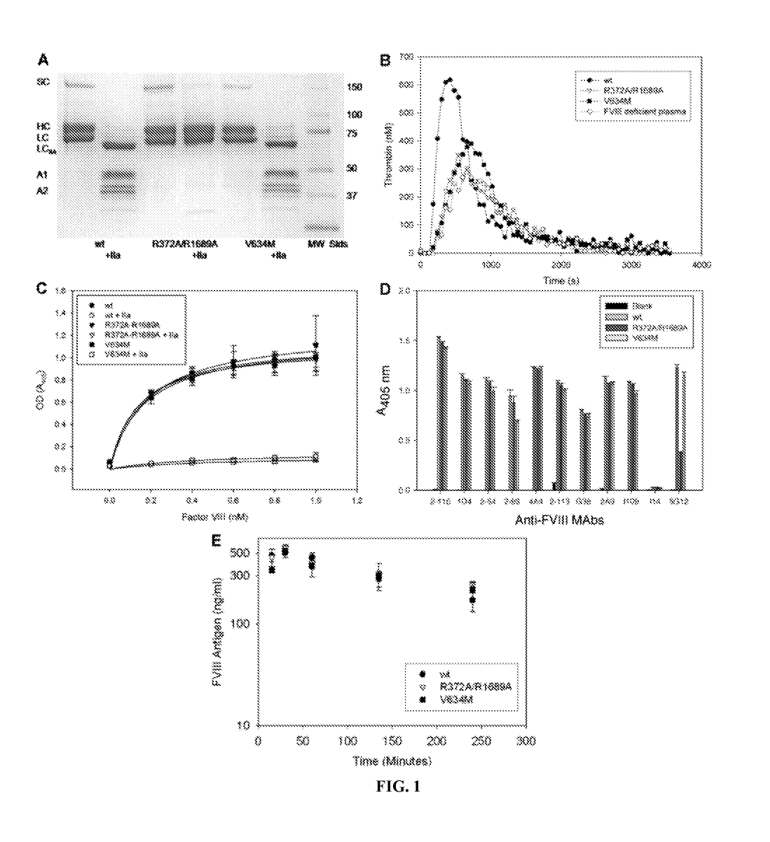

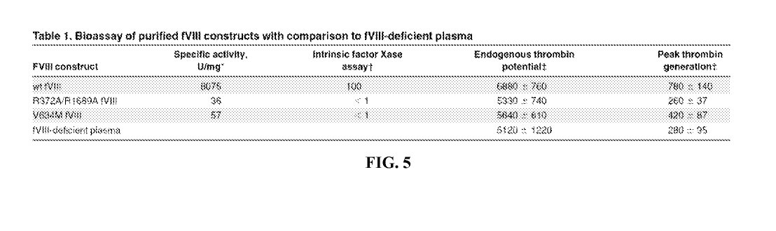

It has been discovered that R372A/R1689A fVIII is marginally less immunogenic and that V634M fVIII was equally immunogenic as wt BDD fVIII in mouse model systems. V634M fVIII is associated with a severe hemophilia A mutation and has <1% of the specific procoagulant of wt fVIII (FIG. 5), yet is cleaved normally (FIG. 1A) and dissociates from VWF after exposure to thrombin (FIG. 1C). The inability of V634M fVIII to function as a procoagulant, while retaining the immunogenic potential of wt fVIII, indicates that the highly immunogenic nature of fVIII is independent of downstream events that lead to thrombin production and the development of an inflammatory milieu.

This conclusion is in sharp contrast to that of Skupsky et al. who found that a heat-inactivated fVIII preparation was less immunogenic than wt fVIII and concluded that the immunogenicity of fVIII was primarily linked to its procoagulant function. See Blood 2009, 114(21):4741-4748. Although there was no apparent reduction in the T-cell epitopes in the heat-inactivated fVIII preparation, there was a significant denaturation of B-cell epitope structure. These properties of heat-denatured fVIII are consistent with the fact that T-cell epitopes are short, linear peptides that are resistant to heat denaturation, whereas most B-cell epitopes are conformationally dependent on the overall fold of a protein. Protein denaturation and loss of antigenic structure of fVIII could lead to procoagulant function-independent reduction in immunogenicity compared with the native protein for several reasons. Antibodies are produced by plasma cells, which are the progeny of a direct differentiation pathway or from a memory B-cell pool that each start with naive B cells. Activation of naïve and memory B cells into their differentiation pathways is initiated by binding of the intact, native antigen to the sIg component of the B-cell receptor. Skupsky et al. found that heat denaturation of fVIII led to destruction of the B-cell epitopes for mAbs ESH4, 1B5, and 3E6, which recognize the phospholipid and VWF-binding region of the fVIII C2 domain, mAbs 2-77 and 3G6, which recognize the so-called nonclassic C2 inhibitor epitope, and Abs 413, 4A4, and 2-76, which recognize an immunodominant inhibitory A2 epitope. Thus, heat denaturation of an antigen could lead to a loss of immunogenicity by destruction of the sIg B-cell epitopes that are required for B-cell activation and differentiation.

B-cell differentiation in response to soluble protein antigens requires CD4+ T-cell help, in which peptide antigen-MCH II complex on the B-cell surface binds to the T-cell receptor on antigen-specific T cells. Antigen-specific T cells are produced from naive T cells after engagement of their T-cell receptors by peptide-MCH II complex on APCs, which include DCs, macrophages, and B cells along with appropriate costimulation. The mannose receptor (CD206) has been implicated in endocytosis of fVIII by DCs, suggesting that intact, nondenatured fVIII may be required for efficient antigen presentation. Binding of fVIII to an additional CD206-independent DC endocytic receptor has been reported. This interaction was blocked by mAb KM33, which recognizes phospholipid binding C1 domain residues 2092-2093. Skupsky et al found that heat denaturation of fVIII led to destruction of the B-cell epitope for the mAb 2A9, an anti-C1 mAb. Thus, naïve C1 structure, which was destroyed by heat treatment in the study by Skupsky et al, may be important for antigen presentation by DCs.

Binding of antigen to high-affinity sIg on B cells leads to antigen presentation at concentrations of free antigen that are significantly lower than those required for presentation by DCs and macrophages, which lack sIg. In addition, DCs engulf intact antigen by macropinocytosis, store it in endocytic compartments, and release it in secondary lymphoid organs, where it binds the sIg on B cells. These results further indicate that naïve antigen is important for efficient antibody production. Thus, the decrease in immunogenicity of fVIII after heat-denaturation observed by Skupsky et al may have resulted from destruction of intrinsic structural elements in fVIII that are independent of its procoagulant function but are required for its immunogenicity. The observation by Skupsky et al. that the immune response to fVIII is decreased in fVIII−/− mice received anticoagulation agents of warfarin or hirudin is consistent with the hypothesis that the coagulation process provides an immunogenic milieu. However, results herein indicate that the immunogenicity of fVIII is predominantly independent of its procoagulant function.

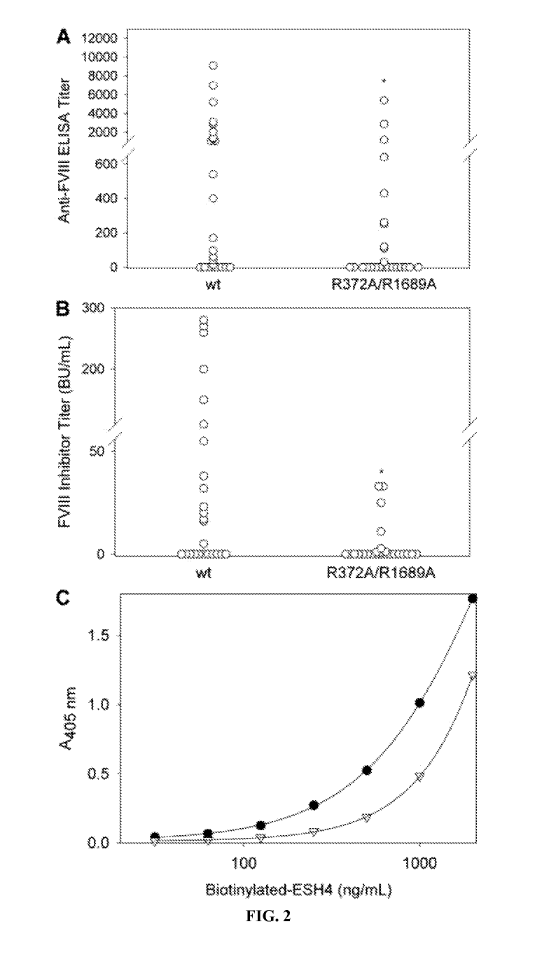

R372A/R1689A fVIII has <1% of the specific procoagulant activity of wt fVIII but, unlike V634M fVIII, does not dissociate from VWF on exposure to thrombin (FIG. 1C). Thus, R372A/R1689A fVIII is a tool to address the question of whether dissociation of activated fVIII from VWF plays a role in the immune response to fVIII. There was a reduction in immunogenicity of R372A/R1689A fVIII compared with wt fVIII in fVIII−/− mice (FIGS. 2A-B and 3). This decrease in immunogenicity may be a result of inhibition of antigen presentation of fVIII when bound to VWF.

The C2 domain and the acidic NH₂-terminal region of the light chain of fVIII determine the high-affinity binding of fVIII to VWF. The binding of fVIII to phospholipid membranes involves an interaction with the C2 domain that overlaps the VWF binding site. A large component of the immune response to fVIII in humans and fVIII−/− mice typically includes inhibitory antibodies directed to this site. Thus, VWF may have the additional anti-immunogenic property of inhibiting recognition of fVIII by B cells that recognize this immunodominant C2 epitope. Although there was a marginal reduction in immunogenicity, all mice treated with the highest dose of R372A/R1689A fVIII developed an immune response. This residual activity could be from the intrinsic dissociation rate of fVIII from VWF in the absence of thrombin or to factors intrinsic to the structure of fVIII that are maintained. Thus, these data suggest that the protective effect of VWF is minor compared with other mechanisms that drive the immunogenicity of fVIII.

Injection of fVIII−/−/VWF−/− mice with wt fVIII or R372A/R1689A fVIII with the use of the dosing schedule for fVIII−/− mice described in FIG. 1 failed to produce an immune response, suggesting that the presence of VWF contributes to the strongly immunogenic nature of fVIII. Alternatively, the greater C57BL/6 background in fVIII−/−/VWF−/− mice may contribute to this difference. VWF potentially brings all fVIII constructs to a hemostatic site even if R372A/R1689A fVIII is not released from the VWF. Cleavage of wt fVIII and V634M fVIII by thrombin may produce an additional marginal increase in immunogenicity relative to R372A/R1689A fVIII. In addition, the circulatory lifetime of fVIII in the presence of normal plasma levels of VWF is similar to that of VWF. In contrast, the circulatory lifetime of fVIII in humans markedly decreases in the absence of VWF. Likewise, the clearance of fVIII in VWF−/− mice is markedly faster than in mice with normal levels of VWF. This result indicates that human fVIII forms a complex with murine VWF after exogenous administration. In addition, these results indicated that the clearance of exogenous human fVIII in humans and mice is largely governed by the clearance of human and murine VWF, respectively. The clearance receptor for VWF has not been identified, although studies in mice indicate that macrophages play a prominent role. As with other blood-born antigens, intravenous injection of human fVIII into fVIII−/− mice results in uptake by the spleen, with preferential uptake by marginal zone macrophages. Overall, these results indicate that in VWF+/+ mice, human fVIII binds circulating VWF and is cleared by splenic macrophages by an unknown VWF receptor, whereas in VWF−/− mice, fVIII is cleared by a different mechanism. Several candidate VWF-independent clearance receptors for fVIII have been identified, including low-density lipoprotein receptor-related protein, the low-density lipoprotein receptor, heparan-sulfate proteoglycans, and the asialoglycoprotein receptor. In the absence of VWF, clearance of fVIII by any 1 of these receptors may lead to degradation of fVIII in a process that does not include antigen presentation. Thus, VWF may decrease the uptake of fVIII by APCs, yet paradoxically may be necessary to prevent clearance of fVIII by pathways that do not promote antigen presentation.

Cross-linking of sIg on B cells is the classic mechanism by which signals are produced that lead to B-cell differentiation into memory and antibody-secreting cells. Yet, if individual B cells have identical sIg molecules on their surface, it is difficult to see how antigens with nonrepetitive structures, such as fVIII, could lead to sIg cross-linking. VWF is a multimer that contains multiple repetitive binding sites for fVIII, which could promote cross-linking of fVIII-specific B-cell receptors leading to anti-fVIII antibody development.

Another possible reason for the striking immunogenicity of fVIII is that intrinsic structural elements, including B-cell or T-cell epitopes, are particularly well recognized by the immune system. FVIII-specific T-cell responses have been readily identified in humans and fVIII−/− mice. Although it has been difficult to identify T-cell epitopes that are clearly associated with fVIII inhibitor formation, Steinitz et al. recently identified 8 dominant T-cell epitopes associated with antibody production in a humanized MHC class II murine hemophilia A model. See Blood, 2012, 119(17):4073-4082. Studies with model small immunogens indicate that nearly the entire surface of a protein is potentially antigenic. Consistent with this, analysis of B-cell epitopes in the C2 domain after immunization of fVIII−/− mice with fVIII found a continuous spectrum of overlapping epitopes. Nonetheless, immunodominant B-cell epitopes in fVIII appear to exist, including the classic and nonclassic C2 domain epitopes and an A2 domain epitope bounded by residues 484-508. Conceivably, the immune response to fVIII initially may be focused on these or other B-cell epitope or unidentified immunodominant T-cell epitopes. The immune response to fVIII then may expand by epitope spreading to produce the observed polyclonal response.

Studies that used structurally intact inactive fVIII molecules indicated that a main component of the immune response to fVIII is independent of its procoagulant function. The immune response is both positively and negatively affected by its association with VWF and may involve intrinsic elements of fVIII structure.

Certain embodiments are a method of improving blood clotting comprising administering a conjugate of a fVIII peptide and a toxin to a subject at risk of developing a fVIII antibody response. In certain embodiments, the subject is a patient diagnosed with Hemophilia A. In certain embodiments, the subject is administered fVIII or a fVIII peptide or derivative in combination or alternation with the conjugate.

In certain embodiments, the conjugate is administered in a pharmaceutical composition. In some embodiments, the composition includes a lipid nanoparticule carrier. In other embodiments, the composition includes a polymeric carrier, including a hydrophilic carrier. In certain embodiments, the pharmaceutical composition includes a carrier to increase cellular uptake.

Decrease in the Immunogenicity of fVIII by Antigen-Specific Deletion of Naïve and Memory fVIII-specific B Cells

The formation of antigen-specific memory B cells plays an essential role in the T helper-cell dependent humoral immune responses. Naïve and memory B cells express antigen-specific sIgs. Targeting fVIII-specific sIgs with fVIII conjugated to a toxin, e.g., saporin, will result in elimination of fVIII-specific naïve and memory B cells. This would prevent de novo immune responses to fVIII and eradicate existing anti-fVIII antibodies.

The potent, intracellular toxin saporin is conjugated to fVIII at a site within a truncated, modified B domain. Saporin is a 30-kDa ribosome-inactivating (RIP) found in soapwort seeds. It, along other RIPs such as ricin and abrin, is a potent intracellular toxin. Saporin typically utilizes cellular entry to exert its function, which is to selectively remove a single adenine from 60S ribosomal RNA and inhibit protein synthesis causing cell death.

The saporin-fVIII conjugate will bind to sIg of fVIII-specific naïve and memory B cells and will be internalized, producing cell death. One can use fVIII that is biotinylated at a specific site in the B domain to produce a conjugate such as a streptavidin-saporin fusion protein. The B domain will be used because proteins (e.g., GFP) can be inserted into this region without loss of functional activity of fVIII. See Li et al., Use of blood outgrowth endothelial cells for gene therapy of hemophilia A, 2002, Blood 99:457-462.

In some embodiments, the presence of anti-fVIII antibodies in a subject is reduced. In certain cases, the reduction can be by about 50%, or by about 60%, or by about 70% or by about 80% or by about 90% or or by about 95% or by about 99% as compared to the subject prior to administration of the conjugate. In certain cases, the formation of fVIII antibodies upon subsequent administration of fVIII or a fVIII peptide or derivative can be prevented or reduced. The formation can be reduced by by about 50%, or by about 60%, or by about 70% or by about 80% or by about 90% or or by about 95% or by about 99% as compared to a subject not administered the conjugate. Antibody production or presence can be measured in plasma of the subject. In some instances, the antibodies are measured using an ELISA or other method known in the art. In certain embodiments, the administration of the conjugate results in an increase in blood clotting.

### EXPERIMENTAL

Characterization of Inactive fVIII Constructs R372A/R1689A fVIII and V634M fVIII

Two inactive BDD fVIII molecules were constructed to investigate the roles of fVIII activation, localization of fVIII at sites of hemostasis and inflammation, and fVIII release from VWF on the immunogenicity of fVIII in murine model systems. R372A/R1689A fVIII lacked thrombin and factor Xa cleavage sites at R372 and R1689. As a result, it did not undergo heavy chain or light cleavage by thrombin (FIG. 1A). The specific procoagulant activity of R372A/R1689A fVIII was <1% of wt fVIII, and it lacked detectable cofactor activity in a purified intrinsic factor Xase assay (FIG. 5). In addition, it did not correct the defect in endogenous thrombin potential or peak thrombin generation of fVIII-deficient plasma in a thrombin generation assay (FIG. 1B; FIG. 5). Cleavage at R1689 in the light chain of fVIII was necessary for its dissociation from VWF. R372A/R1689A fVIII bound normally to VWF, but it did not dissociate from VWF on exposure to thrombin (FIG. 1C). Therefore, it was unlikely that R372A/R1689A fVIII would localize to the phospholipid membrane at a hemostatic site.

V634M fVIII is a severe hemophilia A mutation associated with normal fVIII antigen levels but <1% coagulant activity. Unlike R372A/R1689A fVIII, V634M was cleaved normally by thrombin (FIG. 1A) and dissociated from VWF after exposure to thrombin (FIG. 1C) However, like R372A/R1689A fVIII, it had <1% activity of wt fVIII by 1-stage coagulation assay or by purified intrinsic Xase assay (FIG. 5) and did not correct the defect in thrombin generation of fVIII-deficient plasma (FIG. 1B; FIG. 5). Thus, V634M fVIII should localize to sites of hemostasis but not promote fibrin formation.

During purification, the chromatographic behavior of R372A/R1689A fVIII and V634M fVIII was indistinguishable from wt fVIII indicating that they maintain structural integrity. To examine their structural integrity further, the ability of the constructs to bind to a panel of 11 nonoverlapping mAbs that collectively recognize all the domains of BDD fVIII was investigated by ELISA. R372A/R1689A fVIII and V634M fVIII bound all mAbs similarly to wt fVIII, except for 5G12. 5G12, an anti-A3 mAb, bound to R372A/R1689A but with lower absorbance than wt fVIII and V634M. These results indicated that R372A/R1689A and V634M are structurally intact (FIG. 1D). The clearance of R372A/R1689A, V634M, and wt fVIII was similar in fVIII−/− mice (FIG. 1E), indicating that the potential differential immunogenicity of these constructs was not because of alternative clearance mechanisms.

Immunogenicity of Low-Dose wt fVIII and R372A/R1689A fVIII in fVIII−/− Mice

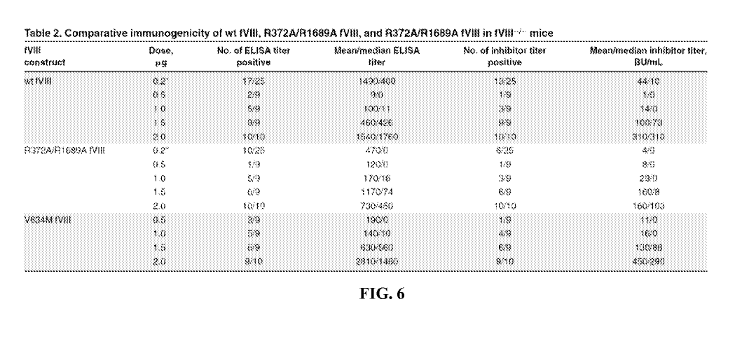

The immunogenicity of wt fVIII and R372A/R1689A fVIII was compared in fVIII−/− mice with the use of doses similar to the dose of fVIII used in humans on the basis of body weight. In this low-dose model, mice received 6 weekly tail vein injections of 0.2 μg, followed by 2 injections of 0.5 μg. For mice injected with wt fVIII, 68% had positive ELISA titers with a mean titer of 1490 and a median titer of 400 (FIG. 2A; FIG. 6). In contrast, R372A/R1689A fVIII produced positive ELISA titers in 40% of mice with a mean titer of 470 and a median titer of 0 (FIG. 2A). The difference in ELISA titers between the 2 groups was significant (P=0.03, Mann-Whitney test). FVIII inhibitor titers in mice injected with wt fVIII displayed a mean of 44 BU/mL and a median of 10 BU/mL compared with a mean of 4.3 BU/mL and a median of 0 in the R372A/R1689A fVIII group (FIG. 2B). The difference in fVIII inhibitor titers between the 2 groups also was significant (P=0.02, Mann-Whitney test). Although R372A/R1689A fVIII, which is not released from VWF after thrombin exposure, was less immunogenic than wt fVIII in this model, it retained significant immunogenicity with 40% of mice showing evidence of an immune response.

Epitope mapping with the use of domain-specific anti-fVIII antibodies found that both wt fVIII and R372A/R1689A fVIII produced polyclonal responses to both heavy and light chain epitopes. Because R372A/R1689A fVIII was not released from VWF after exposure to thrombin, and because VWF bound to the fVIII C2 domain at a site that overlapped the binding site for classic anti-C2 antibodies, it was determined whether VWF shielded the classic C2 epitope from antibody development. FVIII was immobilized on microtiter wells, and the binding of the biotinylated classic anti-C2 mAb, ESH4, was measured in the presence or absence of plasma from immunized fVIII−/− mice by ELISA. In FIG. 2C, the rightward shift of the biotinylated ESH4 binding curve for 1 of the 4 high-titer fVIII inhibitor plasmas shown in FIG. 2B indicated the presence of antibodies directed against classic C2 domain epitopes. Two of the 4 high-titer R372A/R1689A fVIII had anti-classic C2 domain antibodies that were detected with this method. Thus, the inability of fVIII to dissociate from VWF after thrombin cleavage did not protect against formation of classic anti-C2 antibodies.

Dose-Dependent Immunogenicity of wt fVIII, R372A/R1689A fVIII, and V634M fVIII in fVIII−/− Mice

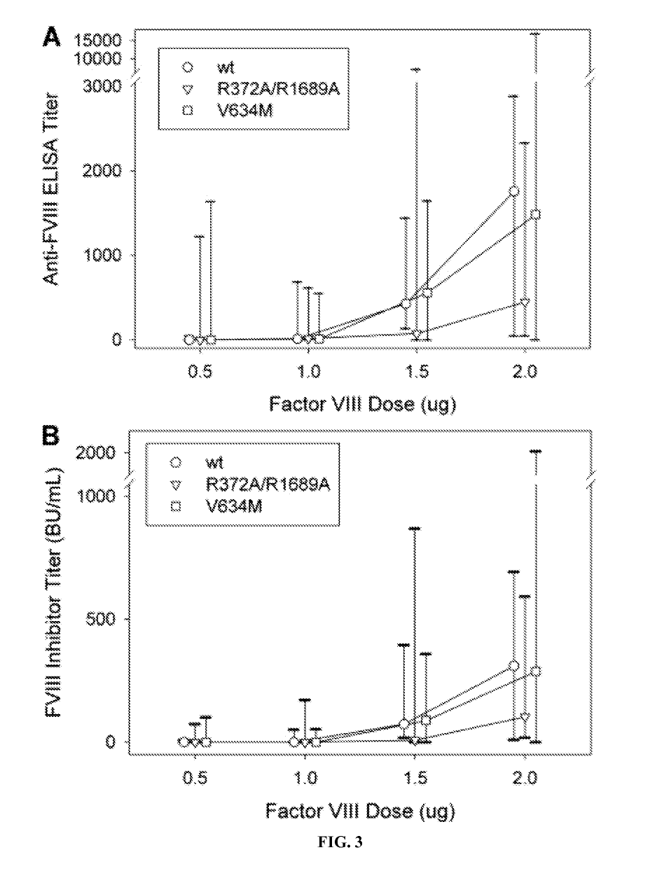

To investigate the role of fVIII function and dose in the immunogenicity of fVIII further, the dose-dependent immunogenicity of wt fVIII with R372A/R1689A fVIII and V634M fVIII was compared Immunogenicity was determined after 4 weekly injections of 0.5, 1.0, 1.5, or 2.0 μg fVIII, followed by an additional injection at twice the nominal dose (FIG. 3; FIG. 6). A dose-dependent increase was observed in total anti-fVIII antibodies measured by ELISA and in the fVIII inhibitor titer for all 3 constructs. The median ELISA titer at 2.0 μg was 1760 for wt fVIII, 450 for R372A/R1689A fVIII, and 1480 for V634M fVIII. The anti-fVIII ELISA titers and fVIII inhibitor titers at each dose were not significantly different between either of the 2 inactive fVIII molecules and wt fVIII (P>0.13 and P>0.31, respectively, Mann-Whitney test). However, a trend was observed toward significance in the decreased immunogenicity observed in the 2.0-μg subgroup, comparing wt fVIII and R372A/R1689A fVIII (P=0.14 for both anti-fVIII ELISA titers and fVIII inhibitors titers, Mann-Whitney test). In addition, no difference was observed in the isotype distribution of IgG1, IgG2a, IgG2b, and IgG3 antibodies in plasmas of mice from the 2.0-μg dosing groups (data not shown). The observation that the inactive R372A/R1689A and V634M fVIII molecules are immunogenic in fVIII−/− mice indicated that fVIII structure not function was the primary driver of immunogenicity. The marginal decreased immunogenicity of R372A/R1689A fVIII compared with wt fVIII suggested that VWF may be partially protective.

Immunogenicity of wt fVIII, R372A/R1689A fVIII, and V634M fVIII in fVIII−/−/VWF−/− Mice

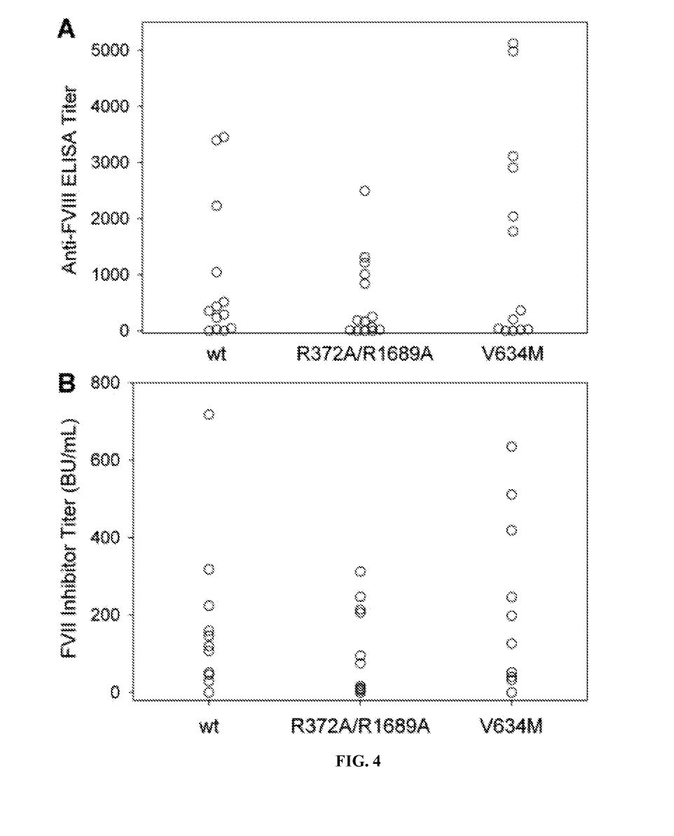

To investigate further the role of VWF in the immunogenicity of fVIII, the immunogenicity of wt fVIII, R372A/R1689A fVIII, and V634M fVIII in fVIII−/−/VWF−/− mice were compared. In initial experiments, using the low-dose wt fVIII regimen, none of 4 fVIII−/−/VWF−/− mice treated with wt fVIII and none of 3 fVIII−/−/VWF−/− mice treated with R372A/R1689A fVIII produced anti-fVIII ELISA titers >40. The lack of an immune response in fVIII−/−/VWF−/− mice contrasted with the immune response to wt fVIII and R372A/R1689A fVIII observed in fVIII−/− mice (FIG. 2A). Therefore, a higher dose regimen that compared wt fVIII, R372A/R1689A fVIII, and V634M was used, consisting of 6 weekly injections of 0.6 μg, followed by 2 additional injections of 1.5 μg. With this regimen, most of the fVIII−/−/VWF−/− mice in all 3 cohorts had detectable anti-fVIII antibodies (FIG. 4A) and fVIII inhibitors (FIG. 4B). Eighty-five percent of the mice had positive ELISA titers in the wt fVIII cohort compared with 79% for R372A/R1689A fVIII and 85% for V634M fVIII (FIG. 4A). The median ELISA titers were similar for each group at 350 for wt fVIII, 180 for R372A/R1689A fVIII, and 360 for V634M fVIII. The inhibitor titers also were similar for each group with a median inhibitor titer of 110 BU/mL for wt fVIII, 46 BU/mL for R372A/R1689A fVIII, and 200 BU/mL for V634M fVIII (FIG. 4B). The differences in anti-fVIII antibody and fVIII inhibitor titers between wt fVIII and R372A/R1689A fVIII and V634M fVIII were not statistically significant. This result was consistent with the conclusion that fVIII structure and not function is the primary determinant of immunogenicity. In addition, the finding that R372A/R1689A fVIII was equally immunogenic to wt fVIII in fVIII−/−/VWF−/− mice indicated that the slightly decreased immunogenicity seen in the fVIII−/− mice was probably because of small protective effect of VWF on presentation of fVIII to the immune system.

### Saporin-fVIII Fusion Protein

One can produce a saporin-fVIII fusion protein consisting of a single polypeptide chain creating a cDNA that consists of the saporin cDNA embedded in the B domain region of fVIII or at the C-terminal end of the fVIII cDNA. In one embodiment, a fVIII-saporin fusion protein can be constructed by encoding a dithiocyclopeptide linker at the fusion site. The dithiocyclopeptide linker contains a thrombin-sensitive sequence and a disulfide bond, allowing for intracellular dissociation of fVIII and saporin. See Chen et al. BioTechniques 49:513-518, 2010.

Alternatively, fVIII and saporin can be joined together using crosslinking agents. FVIII does not have an accessible free cysteine. Thus, the creation of mutant fVIII proteins that contain single free cysteines allows for site-specific linking to saporin. Five B domain-deleted fVIII constructs, R740A/C753 fVIII, S133C fVIII, S367C fVIII, M1711C fVIII, and K2110C fVIII have been constructed. R740A/C753 fVIII has been biotinylated using maleimide-PEG2-biotin, which was followed by conjugation of saporin to this site using streptavidin-saporin.

Alternatively, a saporin-fVIII fusion protein can be prepared by introducing a single free cysteine into saporin, which also does not have accessible free cysteines, and linking the two free cysteines together with a bifunctional crosslinker. A free cysteine is introduced into residue of 255 of saporin as described by Gunhan et al (see Protein Expression and Purification 58 (2008) 203-209). The modified saporin molecule, called C255sap-3, is expressed from E. coli and purified by ion exchange chromatography. The mutants R740A/C753 fVIII, S133C fVIII, S367C fVIII, M1711C fVIII or K2110C fVIII can be modified with the homobifunctional linker dithiobismaleimidoethane (DTME), creating a DTME-fVIII protein containing a single free maleimidyl groups. DTME-fVIII then can be reacted with C255sap-3 to link saporin to the other end of the DTME. The product is fVIII-DTME-saporin.

Further a free amine on fVIII can be targeted either specifically or nonspecifically with a heterobifunctional linker. Free amines in fVIII are conjugated with the heterobifunctional cross-lining agent N-succinimidyl 3-(2-pyridyldithio)proprionate (SPDP). C255sap-3 then is added to the SPDP-fVIII conjugate and cross-links to the 2 pyridylthio sulfthydryl reactive group of SPDP. The product, designated sap-fVIII, is purified using a desalting column.

Alternatively, a construct can be developed by using split intein technology following the protocol described in Ludwig, et al. (2009) Methods in Enzymology. 462:77-96. Briefly, the method includes expressing a fVIII fusion protein with an intein peptide in a host cell, typically a mammalian cell, and expressing a toxin, typically saporin, fusion protein with a intein peptide in a separate cell in which the toxin can be effectively expressed without leading to cell death, such as a bacterial cell. The two intein-containing fusion proteins are mixed and the intein sequence, which is autocatalytic, allows the conjugation of the proteins.

In Vitro Effect of Saporin-fVIII on Memory B Cells in Naïve Mice and in fVIII Inhibitor Mice

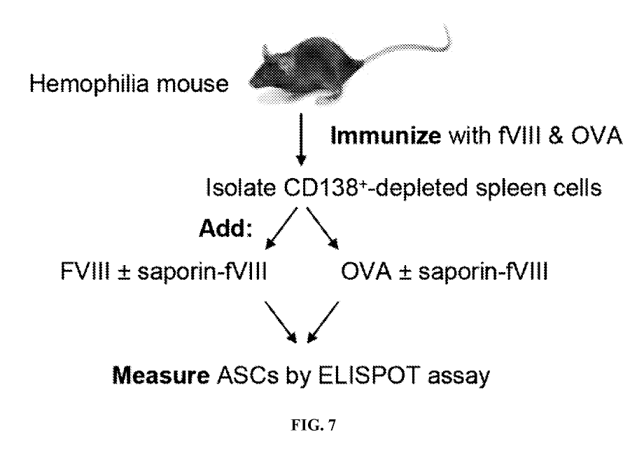

For an overview, see FIG. 7. Mice will be immunized with both fVIII and OVA under conditions that produce an Ab response to both proteins. The ability of splenic memory B cells to be converted to ASCs in response to fVIII, saporin-fVIII, fVIII+saporin-fVIII, OVA and OVA+saporin fVIII will be determined by enzyme-linked immunospot (ELISPOT) assay. The fVIII-specific and OVA-specific memory B cell assays are established methods. Spleen cells are plated on a well containing immobilized fVIII. Cells that secrete anti-fVIII Ab are identified by the spot that forms when the Ab binds fVIII and is detected with a secondary Ab conjugated with horseradish peroxidase.

Mice will be immunized with both adjuvant-free fVIII (three weekly doses of 10 μg/kg by tail vein) and adjuvant free OVA (three weekly intraperitoneal doses of 1000 μg/kg, 500 μg/kg and 500 μg/kg, respectively). The OVA immunization schedule has been shown to produce a measureable Ab response in the absence of adjuvants. Two weeks after the last dose, splenic single cell suspensions will be prepared and depleted of ASCs using anti-CD138 Ab. The resulting spleen cell population includes memory B cells, T cells and APCs. fVIII, saporin-fVIII and OVA will be added to the ASC-deficient spleen cell preparations at concentrations ranging from zero to 0.2 μg/ml, 0.3 μg/ml and 10 μg/ml respectively. Six days later the conversion of memory B cells to ASCs will be determined by anti-fVIII and anti-OVA ELISPOT assay.

This experiment will show that saporin-fVIII specifically blocks the conversion of fVIII memory B cells to ASCs in vitro and is non-toxic to unrelated OVA memory B cells.

E16 hemophilia A mice (donor mice) are immunized with adjuvant-free fVIII consisting four weekly doses of 2 μg by tail vein followed one week later by a single injection of 4 μg. Four weeks later, these mice are then injected by tail vein either 0.4 μg saporin or 2.4 μg of sap-fVIII, representing equal molar amounts of the proteins. One week after injection the spleens from the mice are removed and single cell suspensions are prepared. CD138⁺ antibody secreting spleen cells are removed by magnetic bead immunodepletion. The resulting CD138⁻ spleen cell preparation containing memory B cells is injected by tail vein into recipient E16 hemophilia A mice previously subjected to 530 rads of sub-lethal radiation. Two days after the injection of spleen cells, recipient mice are immunized with 0.5 μg or 2 μg fVIII to stimulate memory B cells derived from the donors. Four weeks later, plasma samples are obtained from the recipients and anti-fVIII titers are measured by ELISA.

The results are as follows:

| | | Recipient | Anti-fVIII |
|---|---|---|---|
| Mouse | Donor Mouse | Mouse | titer |
| 1 | Saporin | 0.5 μg fVIII | 30 |
| 2 | Saporin | 2 μg fVIII | 9000 |
| 3 | Sap-fVIII | 0.5 μg fVIII | 10 |
| 4 | Sap-fVIII | 2 μg fVIII | 30 |

These results show that recipient mice receiving cells from saporin donors produce high titer anti-fVIII antibodies when immunized with a single dose of 2 μg fVIII, indicating the presence of donor-derived fVIII-specific memory B cells in these mice. In contrast, recipient mice receiving cells from sap-fVIII donors do not produce high titer anti-fVIII antibodies, indicating the memory B cells were eradicated in donor mice prior transfer into recipient mice.

1. A conjugate comprising a factor VIII polypeptide or variant thereof and a toxin.

2. The conjugate of claim 1, wherein the toxin is a polypeptide.

3. The conjugate of claim 1, wherein the toxin is selected from saporin, ricin, and abrin.

4. The conjugate of claim 1, wherein the factor VIII polypeptide is linked to the toxin by a linking group.

5. (canceled)

6. The conjugate of claim 2, wherein the conjugate is a recombinant polypeptide.

7. A nucleic acid encoding a recombinant polypeptide of claim 6.

8. An expression vector comprising a nucleic acid of claim 7.

9. An expression system comprising a vector of claim 8.

10. A method of improving blood clotting comprising administering a conjugate of claim 1 to a subject that has or it as risk of having plasma anti-Factor VIII antibodies, wherein administration is in an effective amount to prevent an adaptive immune response to Factor VIII.

11. The method of claim 10, wherein Factor VIII is administered in combination with or after administering the conjugate of claim 1.

12. The method of claim 11, wherein the subject is diagnosed with Hemophilia A.

13-14. (canceled)


SEQUENCE LISTING (4 sequences)

SEQ ID NO: 1 - protein, 2351 aa, Homo sapiens
MQIELSTCFFLCLLRFCFSATRRYYLGAVELSWDYMQSDLGELPVDARFPPRVPKSFPFNTSVVYKKTLFVEFTDHLFNIAKPRPPWMGLLGPTIQAEVYDTVVITLKNMASHPVSLHAVGVSYWKASEGAEYDDQTSQREKEDDKVFPGGSHTYVWQVLKENGPMASDPLCLTYSYLSHVDLVKDLNSGLIGALLVCREGSLAKEKTQTLHKFILLFAVFDEGKSWHSETKNSLMQDRDAASARAWPKMHTVNGYVNRSLPGLIGCHRKSVYWHVIGMGTTPEVHSIFLEGHTFLVRNHRQASLEISPITFLTAQTLLMDLGQFLLFCHISSHQHDGMEAYVKVDSCPEEPQLRMKNNEEAEDYDDDLTDSEMDVVRFDDDNSPSFIQIRSVAKKHPKTWVHYIAAEEEDWDYAPLVLAPDDRSYKSQYLNNGPQRIGRKYKKVRFMAYTDETFKTREAIQHESGILGPLLYGEVGDTLLIIFKNQASRPYNIYPHGITDVRPLYSRRLPKGVKHLKDFPILPGEIFKYKWTVTVEDGPTKSDPRCLTRYYSSFVNMERDLASGLIGPLLICYKESVDQRGNQIMSDKRNVILFSVFDENRSWYLTENIQRFLPNPAGVQLEDPEFQASNIMHSINGYVFDSLQLSVCLHEVAYWYILSIGAQTDFLSVFFSGYTFKHKMVYEDTLTLFPFSGETVFMSMENPGLWILGCHNSDFRNRGMTALLKVSSCDKNTGDYYEDSYEDISAYLLSKNNAIEPRSFSQNSRHPSTRQKQFNATTIPENDIEKTDPWFAHRTPMPKIQNVSSSDLLMLLRQSPTPHGLSLSDLQEAKYETFSDDPSPGAIDSNNSLSEMTHFRPQLHHSGDMVFTPESGLQLRLNEKLGTTAATELKKLDFKVSSTSNNLISTIPSDNLAAGTDNTSSLGPPSMPVHYDSQLDTTLFGKKSSPLTESGGPLSLSEENNDSKLLESGLMNSQESSWGKNVSSTESGRLFKGKRAHGPALLTKDNALFKVSISLLKTNKTSNNSATNRKTHIDGPSLLIENSPSVWQNILESDTEFKKVTPLIHDRMLMDKNATALRLNHMSNKTTSSKNMEMVQQKKEGPIPPDAQNPDMSFFKMLFLPESARWIQRTHGKNSLNSGQGPSPKQLVSLGPEKSVEGQNFLSEKNKVVVGKGEFTKDVGLKEMVFPSSRNLFLTNLDNLHENNTHNQEKKIQEEIEKKETLIQENVVLPQIHTVTGTKNFMKNLFLLSTRQNVEGSYDGAYAPVLQDFRSLNDSTNRTKKHTAHFSKKGEEENLEGLGNQTKQIVEKYACTTRISPNTSQQNFVTQRSKRALKQFRLPLEETELEKRIIVDDTSTQWSKNMKHLTPSTLTQIDYNEKEKGAITQSPLSDCLTRSHSIPQANRSPLPIAKVSSFPSIRPIYLTRVLFQDNSSHLPAASYRKKDSGVQESSHFLQGAKKNNLSLAILTLEMTGDQREVGSLGTSATNSVTYKKVENTVLPKPDLPKTSGKVELLPKVHIYQKDLFPTETSNGSPGHLDLVEGSLLQGTEGAIKWNEANRPGKVPFLRVATESSAKTPSKLLDPLAWDNHYGTQIPKEEWKSQEKSPEKTAFKKKDTILSLNACESNHAIAAINEGQNKPEIEVTWAKQGRTERLCSQNPPVLKRHQREITRTTLQSDQEEIDYDDTISVEMKKEDFDIYDEDENQSPRSFQKKTRHYFIAAVERLWDYGMSSSPHVLRNRAQSGSVPQFKKVVFQEFTDGSFTQPLYRGELNEHLGLLGPYIRAEVEDNIMVTFRNQASRPYSFYSSLISYEEDQRQGAEPRKNFVKPNETKTYFWKVQHHMAPTKDEFDCKAWAYFSDVDLEKDVHSGLIGPLLVCHTNTLNPAHGRQVTVQEFALFFTIFDETKSWYFTENMERNCRAPCNIQMEDPTFKENYRFHAINGYIMDTLPGLVMAQDQRIRWYLLSMGSNENIHSIHFSGHVFTVRKKEEYKMALYNLYPGVFETVEMLPSKAGIWRVECLIGEHLHAGMSTLFLVYSNKCQTPLGMASGHIRDFQITASGQYGQWAPKLARLHYSGSINAWSTKEPFSWIKVDLLAPMIIHGIKTQGARQKFSSLYISQFIIMYSLDGKKWQTYRGNSTGTLMVFFGNVDSSGIKHNIFNPPIIARYIRLHPTHYSIRSTLRMELMGCDLNSCSMPLGMESKAISDAQITASSYFTNMFATWSPSKARLHLQGRSNAWRPQVNNPKEWLQVDFQKTMKVTGVTTQGVKSLLTSMYVKEFLISSSQDGHQWTLFFQNGKVKVFQGNQDSFTPVVNSLDPPLLTRYLRIHPQSWVHQIALRMEVLGCEAQDLY

SEQ ID NO: 2 - protein, 2332 aa, Homo sapiens
ATRRYYLGAVELSWDYMQSDLGELPVDARFPPRVPKSFPFNTSVVYKKTLFVEFTDHLFNIAKPRPPWMGLLGPTIQAEVYDTVVITLKNMASHPVSLHAVGVSYWKASEGAEYDDQTSQREKEDDKVFPGGSHTYVWQVLKENGPMASDPLCLTYSYLSHVDLVKDLNSGLIGALLVCREGSLAKEKTQTLHKFILLFAVFDEGKSWHSETKNSLMQDRDAASARAWPKMHTVNGYVNRSLPGLIGCHRKSVYWHVIGMGTTPEVHSIFLEGHTFLVRNHRQASLEISPITFLTAQTLLMDLGQFLLFCHISSHQHDGMEAYVKVDSCPEEPQLRMKNNEEAEDYDDDLTDSEMDVVRFDDDNSPSFIQIRSVAKKHPKTWVHYIAAEEEDWDYAPLVLAPDDRSYKSQYLNNGPQRIGRKYKKVRFMAYTDETFKTREAIQHESGILGPLLYGEVGDTLLIIFKNQASRPYNIYPHGITDVRPLYSRRLPKGVKHLKDFPILPGEIFKYKWTVTVEDGPTKSDPRCLTRYYSSFVNMERDLASGLIGPLLICYKESVDQRGNQIMSDKRNVILFSVFDENRSWYLTENIQRFLPNPAGVQLEDPEFQASNIMHSINGYVFDSLQLSVCLHEVAYWYILSIGAQTDFLSVFFSGYTFKHKMVYEDTLTLFPFSGETVFMSMENPGLWILGCHNSDFRNRGMTALLKVSSCDKNTGDYYEDSYEDISAYLLSKNNAIEPRSFSQNSRHPSTRQKQFNATTIPENDIEKTDPWFAHRTPMPKIQNVSSSDLLMLLRQSPTPHGLSLSDLQEAKYETFSDDPSPGAIDSNNSLSEMTHFRPQLHHSGDMVFTPESGLQLRLNEKLGTTAATELKKLDFKVSSTSNNLISTIPSDNLAAGTDNTSSLGPPSMPVHYDSQLDTTLFGKKSSPLTESGGPLSLSEENNDSKLLESGLMNSQESSWGKNVSSTESGRLFKGKRAHGPALLTKDNALFKVSISLLKTNKTSNNSATNRKTHIDGPSLLIENSPSVWQNILESDTEFKKVTPLIHDRMLMDKNATALRLNHMSNKTTSSKNMEMVQQKKEGPIPPDAQNPDMSFFKMLFLPESARWIQRTHGKNSLNSGQGPSPKQLVSLGPEKSVEGQNFLSEKNKVVVGKGEFTKDVGLKEMVFPSSRNLFLTNLDNLHENNTHNQEKKIQEEIEKKETLIQENVVLPQIHTVTGTKNFMKNLFLLSTRQNVEGSYDGAYAPVLQDFRSLNDSTNRTKKHTAHFSKKGEEENLEGLGNQTKQIVEKYACTTRISPNTSQQNFVTQRSKRALKQFRLPLEETELEKRIIVDDTSTQWSKNMKHLTPSTLTQIDYNEKEKGAITQSPLSDCLTRSHSIPQANRSPLPIAKVSSFPSIRPIYLTRVLFQDNSSHLPAASYRKKDSGVQESSHFLQGAKKNNLSLAILTLEMTGDQREVGSLGTSATNSVTYKKVENTVLPKPDLPKTSGKVELLPKVHIYQKDLFPTETSNGSPGHLDLVEGSLLQGTEGAIKWNEANRPGKVPFLRVATESSAKTPSKLLDPLAWDNHYGTQIPKEEWKSQEKSPEKTAFKKKDTILSLNACESNHAIAAINEGQNKPEIEVTWAKQGRTERLCSQNPPVLKRHQREITRTTLQSDQEEIDYDDTISVEMKKEDFDIYDEDENQSPRSFQKKTRHYFIAAVERLWDYGMSSSPHVLRNRAQSGSVPQFKKVVFQEFTDGSFTQPLYRGELNEHLGLLGPYIRAEVEDNIMVTFRNQASRPYSFYSSLISYEEDQRQGAEPRKNFVKPNETKTYFWKVQHHMAPTKDEFDCKAWAYFSDVDLEKDVHSGLIGPLLVCHTNTLNPAHGRQVTVQEFALFFTIFDETKSWYFTENMERNCRAPCNIQMEDPTFKENYRFHAINGYIMDTLPGLVMAQDQRIRWYLLSMGSNENIHSIHFSGHVFTVRKKEEYKMALYNLYPGVFETVEMLPSKAGIWRVECLIGEHLHAGMSTLFLVYSNKCQTPLGMASGHIRDFQITASGQYGQWAPKLARLHYSGSINAWSTKEPFSWIKVDLLAPMIIHGIKTQGARQKFSSLYISQFIIMYSLDGKKWQTYRGNSTGTLMVFFGNVDSSGIKHNIFNPPIIARYIRLHPTHYSIRSTLRMELMGCDLNSCSMPLGMESKAISDAQITASSYFTNMFATWSPSKARLHLQGRSNAWRPQVNNPKEWLQVDFQKTMKVTGVTTQGVKSLLTSMYVKEFLISSSQDGHQWTLFFQNGKVKVFQGNQDSFTPVVNSLDPPLLTRYLRIHPQSWVHQIALRMEVLGCEAQDLY

SEQ ID NO: 3 - protein, 253 aa, Saponaria officinalis
VTSITLDLVNPTAGQYSSFVDKIRNNVKDPNLKYGGTDIAVIGPPSKEKFLRINFQSSRGTVSLGLKRDNLYVVAYLAMDNTNVNRAYYFKSEITSAELTALFPEATTANQKALEYTEDYQSIEKNAQITQGDKSRKELGLGIDLLLTFMEAVNKKARVVKNEARFLLIAIQMTAEVARFRYIQNLVTKNFPNKFDSDNKVIQFEVSWRKISTAIYGDAKNGVFNKDYDFGFGKVRQVKDLQMGLLMYLGKPK

SEQ ID NO: 4 - protein, 2311 aa, Homo sapiens
GELPVDARFPPRVPKSFPFNTSVVYKKTLFVEFTDHLFNIAKPRPPWMGLLGPTIQAEVYDTVVITLKNMASHPVSLHAVGVSYWKASEGAEYDDQTSQREKEDDKVFPGGSHTYVWQVLKENGPMASDPLCLTYSYLSHVDLVKDLNSGLIGALLVCREGSLAKEKTQTLHKFILLFAVFDEGKSWHSETKNSLMQDRDAASARAWPKMHTVNGYVNRSLPGLIGCHRKSVYWHVIGMGTTPEVHSIFLEGHTFLVRNHRQASLEISPITFLTAQTLLMDLGQFLLFCHISSHQHDGMEAYVKVDSCPEEPQLRMKNNEEAEDYDDDLTDSEMDVVRFDDDNSPSFIQIRSVAKKHPKTWVHYIAAEEEDWDYAPLVLAPDDRSYKSQYLNNGPQRIGRKYKKVRFMAYTDETFKTREAIQHESGILGPLLYGEVGDTLLIIFKNQASRPYNIYPHGITDVRPLYSRRLPKGVKHLKDFPILPGEIFKYKWTVTVEDGPTKSDPRCLTRYYSSFVNMERDLASGLIGPLLICYKESVDQRGNQIMSDKRNVILFSVFDENRSWYLTENIQRFLPNPAGVQLEDPEFQASNIMHSINGYVFDSLQLSVCLHEVAYWYILSIGAQTDFLSVFFSGYTFKHKMVYEDTLTLFPFSGETVFMSMENPGLWILGCHNSDFRNRGMTALLKVSSCDKNTGDYYEDSYEDISAYLLSKNNAIEPRSFSQNSRHPSTRQKQFNATTIPENDIEKTDPWFAHRTPMPKIQNVSSSDLLMLLRQSPTPHGLSLSDLQEAKYETFSDDPSPGAIDSNNSLSEMTHFRPQLHHSGDMVFTPESGLQLRLNEKLGTTAATELKKLDFKVSSTSNNLISTIPSDNLAAGTDNTSSLGPPSMPVHYDSQLDTTLFGKKSSPLTESGGPLSLSEENNDSKLLESGLMNSQESSWGKNVSSTESGRLFKGKRAHGPALLTKDNALFKVSISLLKTNKTSNNSATNRKTHIDGPSLLIENSPSVWQNILESDTEFKKVTPLIHDRMLMDKNATALRLNHMSNKTTSSKNMEMVQQKKEGPIPPDAQNPDMSFFKMLFLPESARWIQRTHGKNSLNSGQGPSPKQLVSLGPEKSVEGQNFLSEKNKVVVGKGEFTKDVGLKEMVFPSSRNLFLTNLDNLHENNTHNQEKKIQEEIEKKETLIQENVVLPQIHTVTGTKNFMKNLFLLSTRQNVEGSYDGAYAPVLQDFRSLNDSTNRTKKHTAHFSKKGEEENLEGLGNQTKQIVEKYACTTRISPNTSQQNFVTQRSKRALKQFRLPLEETELEKRIIVDDTSTQWSKNMKHLTPSTLTQIDYNEKEKGAITQSPLSDCLTRSHSIPQANRSPLPIAKVSSFPSIRPIYLTRVLFQDNSSHLPAASYRKKDSGVQESSHFLQGAKKNNLSLAILTLEMTGDQREVGSLGTSATNSVTYKKVENTVLPKPDLPKTSGKVELLPKVHIYQKDLFPTETSNGSPGHLDLVEGSLLQGTEGAIKWNEANRPGKVPFLRVATESSAKTPSKLLDPLAWDNHYGTQIPKEEWKSQEKSPEKTAFKKKDTILSLNACESNHAIAAINEGQNKPEIEVTWAKQGRTERLCSQNPPVLKRHQREITRTTLQSDQEEIDYDDTISVEMKKEDFDIYDEDENQSPRSFQKKTRHYFIAAVERLWDYGMSSSPHVLRNRAQSGSVPQFKKVVFQEFTDGSFTQPLYRGELNEHLGLLGPYIRAEVEDNIMVTFRNQASRPYSFYSSLISYEEDQRQGAEPRKNFVKPNETKTYFWKVQHHMAPTKDEFDCKAWAYFSDVDLEKDVHSGLIGPLLVCHTNTLNPAHGRQVTVQEFALFFTIFDETKSWYFTENMERNCRAPCNIQMEDPTFKENYRFHAINGYIMDTLPGLVMAQDQRIRWYLLSMGSNENIHSIHFSGHVFTVRKKEEYKMALYNLYPGVFETVEMLPSKAGIWRVECLIGEHLHAGMSTLFLVYSNKCQTPLGMASGHIRDFQITASGQYGQWAPKLARLHYSGSINAWSTKEPFSWIKVDLLAPMIIHGIKTQGARQKFSSLYISQFIIMYSLDGKKWQTYRGNSTGTLMVFFGNVDSSGIKHNIFNPPIIARYIRLHPTHYSIRSTLRMELMGCDLNSCSMPLGMESKAISDAQITASSYFTNMFATWSPSKARLHLQGRSNAWRPQVNNPKEWLQVDFQKTMKVTGVTTQGVKSLLTSMYVKEFLISSSQDGHQWTLFFQNGKVKVFQGNQDSFTPVVNSLDPPLLTRYLRIHPQSWVHQIALRMEVLGCEAQDLY